(19) United States
(12) Patent Application Publication
(10) Pub. No.: US 2019/0000741 A1
(43) Pub. Date: Jan. 3, 2019

(54) IOTA CARRAGEENAN-MULTI-VALENT CATION ALGINATE BINDER COMPOSITION

(71) Applicant: FMC Corporation, Phiadelphia, PA (US)

(72) Inventors: Amruta Antala, Ewing, NJ (US); Thomas Scott Campbell, Flemington, NJ (US)

(21) Appl. No.: 16/066,766

(22) PCT Filed: Jan. 13, 2017
(86) PCT No.: PCT/US17/13311

§ 371 Date: Jun. 28, 2018

Related U.S. Application Data
(60) Provisional application No. 62/278,784, filed on Jan. 14, 2016.

(51) Int. Cl. A61K 8/73; A61K 8/9717; A61K 8/21; A61Q 11/00

(52) U.S. Cl. CPC A61K 8/733; A61K 8/9717; A61K 2800/592; A61Q 11/00; A61K 2800/524; A61K 8/21

(57) ABSTRACT

A toothpaste composition comprising a binder composition wherein said binder composition comprises (a) alginate comprising a polyvalent cation alginate and (b) carrageenan comprising iota carrageenan. Such binder composition provides desirable rheological properties coupled with unexpectedly enhanced processability and visual characteristics.

### FIELD OF THE INVENTION

The present invention is directed to a toothpaste composition comprising a binder composition wherein said binder composition comprises (a) alginate comprising a polyvalent cation alginate and (b) carrageenan comprising iota carrageenan. Such binder composition provides desirable rheological properties coupled with unexpectedly enhanced processability and visual characteristics.

### BACKGROUND OF THE INVENTION

Toothpastes are used by consumers as an aid to remove dental plaque, a deposit which builds up on teeth and which is formed from an accumulation of bacteria and bacterial byproducts. Multibenefit and antibacterial toothpastes can also aid consumers by preventing an inflammation of the gums, known as gingivitis. If left untreated, this can become a more serious infection known as periodontitis. Gingivitis and periodontitis are major causes of tooth loss in adults.

In order to satisfy consumer expectations, toothpaste compositions need to possess certain physical properties to which the consumer is accustomed. These properties are required to provide a toothpaste that has appealing taste, has good cleansing effect, is easy to rinse, has excellent mouth feel, and has physical stability. Toothpaste compositions with acceptable physical stability do not exhibit phase separation such as water or flavor separation. The appearance of the paste as it comes out of the dispenser is also considered important. It should appear smooth and have a pleasant sheen or glossy appearance.

In order to achieve many of these properties, toothpastes should have a consumer acceptable viscosity and pseudoplastic nature in order to be easily dispensed when the tube is actuated. The toothpaste should also recover from this shear in a timeframe that prevents the paste from sinking into the toothbrush bristles. These qualities are in turn influenced by the selection of raw materials, manufacturing procedure and quality control.

Toothpaste compositions typically contain a polishing agent or abrasive, a humectant, a binder or thickener, a surface active agent or surfactant, and water, as well as materials that provide therapeutic or cosmetic benefits, such as fluorides, flavorings, and sweeteners. Binders in toothpaste play a crucial role in sample texture as they control or modify toothpaste rheology in terms of viscosity, pseudoplasticity, and yield value. Binders will often be used in combination to achieve a desired consistency.

Among the materials which have been employed in the past to bind toothpaste formulations are carrageenans. Unfortunately, while carrageenans are effective to thicken toothpastes as well as to effectively suspend dispersed solid polishing agents, their ability to act as binders decreases when they are subjected to mechanical working at temperatures below their gel-sol transition point as such activity will result in a decrease in the viscosity of the formulation. As is noted in U.S. Pat. No. 4,604,280 (Scott), even relatively minor working, such as that experienced when the toothpaste is pumped or otherwise conveyed at room temperature can cause substantial decreases in viscosity. While viscosity decreases can be minimized by working the formulation at temperatures above its gel-sol transition temperature, as is pointed out by U.S. Pat. No. 4,604,280, in conventional plants for the manufacture of toothpastes, this may often be impractical or difficult because there may often be delays between times when a dentifrice formulation is manufactured and when in is ready for filling into dispensing containers. Further, maintaining an elevated temperature employing conventional open mixing heaters or similar devices may result in local overheating and aeration of the paste with the result that losses of moisture and/or volatile flavor components may occur. U.S. Pat. No. 4,604,208 proposes to overcome these difficulties by the use of microwave radiation.

It is noteworthy that US 4,604,280 further indicates that the addition of calcium salts to carrageenans, particularly to iota carrageenan, can greatly increase the gel-sol transition temperature, making the handling of such a formulation even more difficult in industrial settings. Specifically, U.S. Pat. No. 4,604,280 teaches that, with respect to iota carrageenan, an increase in the calcium ion content from 0 to 1% may increase the gelling temperature from about 44° C. to 72° C. Accordingly, this patent teaches that a mixture of lambda carrageenan with kappa carrageenan is preferred as such mixture exhibits a much lower increase in gel-sol transition temperature.

Unfortunately, as is disclosed in U.S. Pat. No. 6,162,418 (Randive et al) toothpaste formulations based upon kappa carrageenan may tend to harden during storage and are more likely to have syneresis issues (i.e., they are more likely to have water separate from the gel) than do gels based upon iota carrageenan.

A second class of materials which have been employed in the past to bind toothpaste formulations are alginates. However, as is noted in WO 2015/109511 (Shi et al), the use of monovalent cation alginates such as sodium alginate tends to result in low viscosity, drippy formulations; whereas the use of multivalent cation alginates such as calcium alginate tends to confer a stringy texture which is unacceptable for many toothpaste applications. In order to overcome these shortcomings, WO 2015/109511 proposes to employ a binder comprising a sodium calcium alginate wherein the weight ratio of sodium to calcium is from 7:1 to 4:1; and is most typically about 84:16.

Somewhat similarly, WO 2015/039277 (Shi et al) discloses a toothpaste binder composition comprising a blend of sodium alginate with calcium alginate. However, WO 2015/039277 indicates that discoloration begins to become a problem when the weight ratio of sodium:calcium in the alginate gel is less than 80:20.

Consequently, it is entirely unexpected that a toothpaste binder formulation based upon a mixture comprising (a) iota carrageenan; and (b) a polyvalent cation-substituted alginate, particularly a calcium-substituted, alginate, would exhibit desirable viscosity even if processed at lower temperature; coupled with desirable visual properties even when alginates having lower monovalent cation:divalent cation ratios are employed.

### SUMMARY OF THE INVENTION

The present invention is directed to a toothpaste composition comprising a binder composition wherein said binder composition comprises (a) alginate comprising a polyvalent cation alginate and (b) carrageenan comprising iota carrageenan.

### DETAILED DESCRIPTION OF THE INVENTION

The present invention is directed to a toothpaste composition comprising a binder composition wherein said binder composition comprises (a) alginate comprising a polyvalent cation alginate and (b) carrageenan comprising iota carrageenan.

Polyvalent cations which can be employed in the alginate component include calcium, strontium, barium, zinc, iron, manganese, copper, cobalt or nickel or combinations thereof; with calcium being typically employed.

The alginate component typically comprises monovalent cations as well as polyvalent catons. Monovalent cations which can be employed in the alginate component of the binder compositions employed in the practice of this invention include sodium, potassium, ammonium and mixtures thereof; with sodium being typically employed.

In certain embodiments, the monovalent cation will comprise sodium and the polyvalent cation will comprise calcium.

The weight percentage of polyvalent cation (based upon the total weight of monovalent cation plus polyvalent cation) in the alginates employed in the practice of the current invention may range from 10% to 100%; typically, the weight percent of the polyvalent cation will comprise at least 20%; more typically at least 30%, based upon the total weight of cations present in the alginate component of the binder.

In certain embodiments, the alginate will comprise only a polyvalent cation alginate, typically calcium alginate. In other embodiments the alginate component will comprise an alginate comprising both monovalent cations and polyvalent cations, typically sodium calcium alginate. In still other embodiments, the alginate component will comprise a mixture of a polyvalent cation alginate and a monovalent cation alginate, typically a mixture of sodium alginate and calcium alginate.

Polyuronates such as alginate can also be classified by their tendency to gel vs. viscosify when activated with a divalent cation such as calcium. This weight ratio of guluronic acid to mannuronic acid is referred to as the G:M ratio. In general, a ratio greater than 1:1 indicates an alginate with higher gelling potential, although as will be recognized by those of skill in the art, this will be influenced by the percentage of G-G linkages present. The alginate component in this invention typically has a G:M ratio of less than 1:1, and more typically possesses a G:M ratio of between 2:3 and 1:3.

The alginates employed in this invention may be of low viscosity, medium viscosity or high viscosity. With respect to alginates, he term “high viscosity” means having a viscosity of more than 400 mPas,; “medium viscosity” means having a viscosity of 200 to 400 mPas; and “low viscosity” means having a viscosity of less than 200 mPas when measured at 1% in water at 20° C. using Brookfield type RV (e.g. RVT, RVF, RVTDV) with Brookfield RV spindle 2.

The carrageenan component of the binder composition employed in the toothpastes of this invention comprises iota carrageenan. Typically the carrageenan comprises between 50% and 100% by weight iota carrageenan, with the reminder comprising other forms of carrageenan, typically lambda, kappa and/or kappa-2 carrageenan. The carrageenans employed may be of high viscosity, medium viscosity or of low viscosity. With respect to carrageenans, he term “high viscosity” means having a water viscosity of at least about 50 cps, “medium viscosity” means having a water viscosity of about 40 to 50 cps, and “low viscosity” means having a water viscosity of less than 40 cps measured as a 1.5% solution of carrageenan in water at 75° C.

The carrageenans typically employed are typically monovalent salts, more typically sodium and/or potassium salts; although such component may comprise minor amounts of polyvalent cations as well.

The weight ratio of alginate to carrageenan employed in the binders of the toothpastes of this invention will typically range between 1:10 and 1:1, is typically between 1:3 and 1:1, and is more typically 1:2.

The toothpaste composition of the present invention also comprises water, and may further comprise one or more abrasives, humectants, surfactants/foaming agents, a fluoride source, a sweetening agent, flavor and may further comprise a whitening agent, preservative and/or sensitivity agent. Typically, such composition will comprise a) between 0.1% and 2% binder composition; b) between 1% and 50% water; c) between 0% and 50% abrasive; d) between 0% and 40% humectant; e) between 0% and 3% surfactant/foaming agent; f) an appropriate regulated concentration of a fluoride source; g) between 0% and 1% sweetening agent; h) between 0% and 2% flavor; i) a cosmetically efficacious concentration of whitening agent; j) between 0% and 2% preservative; and k) between 0% and 10% sensitivity agent; wherein all such percentages are by weight based upon the total weight of the composition.

Abrasives which may be employed include calcium-based polishing agents, such as dicalcium phosphate dihydrate (generally known as dicalcium phosphate), tricalcium phosphate, calcium carbonate (such as limestone, natural chalk, or precipitated chalk), calcium pyrophosphate, sodium metaphosphate; amorphous silica; crystalline silica; precipitated silica; complex aluminosilicate; aluminum hydroxide; aluminosilicates, bentonite, talc, aluminum oxide, silica xerogels, bicarbonates and mixtures thereof.

The toothpaste compositions of the invention further include one or more humectants. Examples of suitable humectants include polyhydric alcohols (polyols) such as propylene glycol, glycerin, sorbitol, xylitol or low molecular weight polyethyleneglycols (PEGS). In various embodiments, humectants can prevent hardening of paste or gel compositions upon exposure to air, improve surface appearance of the paste and help to provide suitable mouthfeel.

The toothpaste compositions of the invention can further include one or more surfactants/foaming agents. Surfactants useful for the present invention include, without limitation anionic, nonionic, and amphoteric surfactants. Suitable anionic surfactants include, for example, water-soluble salts of C₈₋₂₀ alkyl sulfates, sulfonated monoglycerides of C₈₋₂₀ fatty acids, sarcosinates and taurates; for example sodium lauryl sulfate, sodium coconut monoglyceride sulfonate, sodium lauryl sarcosinate, sodium lauryl isoethionate, sodium laureth carboxylate and sodium dodecylbenzenesulfonate, and mixtures thereof. Suitable nonionic surfactants include, for example, poloxamers, polyoxyethylene sorbitan esters, fatty alcohol ethoxylates, alkylphenol ethoxylates, tertiary amine oxides, tertiary phosphine oxides, dialkyl sulfoxides, and mixtures thereof. In one embodiment, the toothpaste comprises sodium lauryl sulfate, for example in an amount of from 1% to 3%.

The toothpaste compositions of the present invention may also contain a fluoride source—i.e. a fluoride-containing compound having a beneficial effect on the care and hygiene of the oral cavity, e g diminution of enamel solubility in acid and protection of the teeth against decay. Examples of suitable fluoride sources include sodium fluoride, stannous fluoride, amine fluoride, and sodium monofluorophosphate. The appropriate level of fluoride will depend on the particular application.

In some embodiments described above, the toothpaste compositions of the invention can further include one or more sweetening agents, flavoring agents and coloring agents. Any suitable flavoring or sweetening material maybe employed. Examples of suitable flavoring constituents include flavoring oils, e.g. oil of spearmint, peppermint, wintergreen, clove, sage, eucalyptus, marjoram, cinnamon, lemon, and orange, and methyl salicylate. Suitable sweetening agents include sucrose, lactose, maltose, xylitol, sodium cyclamate, saccharine and the like. Suitably, flavor and sweetening agents may each or together comprise from about 0.1% to 5% more of the oral care composition. In some embodiments, the toothpaste compositions of the invention include one or more flavoring agents in an amount of from 0.5% to 2.0%.

The toothpaste compositions described herein may further comprise antimicrobial agents, for example Triclosan, chlorhexidine, copper-, zinc- and stannous salts such as zinc citrate, zinc sulphate, zinc glycinate, sodium zinc citrate and stannous pyrophosphate, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds such as 2,2′ methylenebis-(4-chloro-6-bromophenol). In addition, various other materials may be incorporated in the compositions of this invention such as whitening agents, including peroxides, preservatives, and potassium salts for the treatment of dental hypersensitivity such as potassium nitrate. These agents, when present are incorporated in the compositions of the present invention in amounts which do not substantially adversely affect the properties and characteristics desired.

Toothpaste compositions can be prepared using either the hot process or the ambient process, and either a batch process or a continuous process may be used. The ambient process is sometimes called the cold process. The hot process is described, for example, in Scott, U.S. Pat. No. 4,353,890, and Ballard, U.S. Pat. No. 6,187,293, the disclosures of which are incorporated herein by reference. A continuous process for the manufacture of toothpaste is disclosed, for example, in Ballard, U.S. Pat. No. 6,187,293, the disclosure of which is incorporated herein by reference. A continuous process for the manufacture of toothpaste is also disclosed in Catiis, U.S. Pat. No. 5,236,696.

It is to be understood that each component, compound, substituent, or parameter disclosed herein is to be interpreted as being disclosed for use alone or in combination with one or more of each and every other component, compound, substituent, or parameter disclosed herein.

It is also to be understood that each amount/value or range of amounts/values for each component, compound, substituent, or parameter disclosed herein is to be interpreted as also being disclosed in combination with each amount/value or range of amounts/values disclosed for any other component(s), compounds(s), substituent(s), or parameter(s) disclosed herein and that any combination of amounts/values or ranges of amounts/values for two or more component(s), compounds(s), substituent(s), or parameters disclosed herein are thus also disclosed in combination with each other for the purposes of this description.

It is further understood that each lower limit of each range disclosed herein is to be interpreted as disclosed in combination with each upper limit of each range disclosed herein for the same component, compounds, substituent, or parameter. Thus, a disclosure of two ranges is to be interpreted as a disclosure of four ranges derived by combining each lower limit of each range with each upper limit of each range. A disclosure of three ranges is to be interpreted as a disclosure of nine ranges derived by combining each lower limit of each range with each upper limit of each range, etc. Furthermore, specific amounts/values of a component, compound, substituent, or parameter disclosed in the description or an example is to be interpreted as a disclosure of either a lower or an upper limit of a range and thus can be combined with any other lower or upper limit of a range or specific amount/value for the same component, compound, substituent, or parameter disclosed elsewhere in the application to form a range for that component, compound, substituent, or parameter.

### EXAMPLES

The following examples are provided to illustrate the invention in accordance with the principles of this invention, but are not to be construed as limiting the invention in any way except as indicated in the appended claims.

### Example 1

The following toothpaste compositions were prepared by blending the following ingredients:

| | Grams per |
|---|---|
| Ingredient | kilogram |
| Na Saccharine | 3.8 |
| NaF | 3.8 |
| Sorbitol | 320.5 |
| Glycerin | 320.5 |
| Hydrocolloid | 12.8 |
| (as specified below) | |

a) Sodium calcium alginate having a Na:Ca weight ratio of 64:36; (Comparative Experiment 1A);
b) Iota carrageenan low viscosity; (Comparative Experiment 1B);
c) Iota carrageenan high viscosity; (Comparative Experiment 1C); and
d) Equal amounts of sodium calcium alginate having a Na:Ca weight ratio of 64:36; sodium iota carrageenan and of high mw iota carrageenan; (Example 1).

The compositions (in an amount equivalent to 6 grams of hydrocolloid) were added to 594 mL of deionized water and stirred. The composition of Comparative Experiment A was clear, as was expected as alginates having a high sodium content are known to be water soluble. The compositions of Comparative Experiments B and C were cloudy suspensions, which was expected as carrageenans are typically water insoluble at room temperature. Surprisingly, the blend of Example 1 was clear, despite comprising a high carrageenan content.

### Example 2

Toothpaste formulations having the following composition (by weight percent) were prepared based upon the following recipe:

| | Grams per |
|---|---|
| Ingredient | kilogram |
| Deionized Water | 338.5 |
| Na Saccharine | 3.8 |
| NaF | 3.8 |
| Sorbitol | 320.5 |
| Glycerin | 320.5 |
| Hydrocolloid | 12.8 |

Such formulations were prepared employing the following process:

A premix was prepared by mixing the deionized water with Na Sacchrin and NaF.
The hydrocolloid was added to the glycerin and mixed for 1 minute in a Thermomix mixer.
The sorbitol was added to the mixture and blended for 5 minutes.
The premix was added to the hydrocolloid mix and mixed for 15 minutes at 100° F.
An 8 ounce polypropylene jar was filled with the blended mixture.

The hydrocolloids employed were as follows:

### Ca Alginate

### 64:36 Na:Ca Alginate

### 85:15 Na:Ca Alginate

### Low Viscosity Na Alginate

### High Viscosity Na Alginate

Low Viscosity Na iota carrageenan
High viscosity iota carrageenan

The viscosity of the resultant formulation was measured at a 0.1/sec shear rate and a 1.0/sec shear rate using a stress-controlled AR1500ex rheometer. A 4 cm stainless steel parallel plate with peltier plate base for temperature control was utilized with a 1000 micrometer gap height between plates. The shear rate was varied from 0.01 to 100.0/s over the course of 300 seconds, and data taken using a logarithmic sampling mode. The result of such testing (in cps) is presented in the following Table and provides a view both of viscosity and shear thinning nature of the solutions:

| Example (EX) | | 0.1/sec. | 1.0/sec. |
|---|---|---|---|
| or Comparative | | Shear Rate | Shear Rate |
| Experiment (CE) | Hydrocolloid | (cps) | (cps) |
| 2CE-1 | Ca Alginate | 9,035 | 7,315 |
| 2CE-2 | 64:36 Na:Ca Alginate | 35,030 | 24,690 |
| 2CE-3 | 85:15 Na:Ca Alginate | 29,100 | 22,450 |
| 2CE-4 | Low Visc. Na Alginate | 174 | 183 |
| 2CE-5 | High Visc. Na Alginate | 27,070 | 21,570 |
| 2CE-6 | Low Visc. I-Carrageenan | 391,300 | 66,130 |
| 2CE-7 | High Visc I-Carrageenan | 48,300 | 11,410 |
| 2CE-8 | 50% Low Visc. | 202,900 | 35,320 |
| | I-Carrageenan + | | |
| | 50% High Visc. | | |
| | I-Carrageenan | | |
| 2-EX1 | 34% Ca Alginate + | 447,700 | 71,040 |
| | 66% Low | | |
| | Visc. I-Carrageenan | | |
| 2-CE-9 | 34% Low Visc. | 32,100 | 7,039 |
| | Na Alginate + | | |
| | 66% Low | | |
| | Visc. I-Carrageenan | | |
| 2-CE-10 | 34% High Mw | 201,500 | 43,650 |
| | Na Alginate + | | |
| | 66% Low | | |
| | Visc. I-Carrageenan | | |
| 2-EX-2 | 33% Low Visc. | 334,300 | 54,930 |
| | I-Carrageenan + | | |
| | 33% High Visc. | | |
| | I-Carrageenan + | | |
| | 34% 64:36 | | |
| | Na:Ca Alginate | | |
| 2-EX-3 | 33% Low Visc. | 246,600 | 44,960 |
| | I-Carrageenan + | | |
| | 33% High Visc. | | |
| | I-Carrageenan + | | |
| | 34% 85:15 | | |
| | Na:Ca Alginate | | |

The above results indicate that combinations comprising calcium alginate exhibited increased viscosities (relative to the carrageenan component alone); in contrast, blends of sodium alginate with carrageenan exhibited reduced viscosities (relative to the carrageenan component alone).

### Example 3

The long term stability of several calcium carbonate formulations was evaluated as described below. Toothpaste formulations having the following composition (by weight percent) were prepared based upon the following recipe:

| Ingredients | % Weight |
|---|---|
| Precipitated Calcium | 46.00% |
| Carbonate | |
| Deionized (DI) Water | 21.94% |
| Na Monofluorophosphate | 0.76% |
| (NAFP) | |
| Na Saccharin | 0.20% |
| Na benzoate | 0.30% |
| Hydrocolloid | 0.80% |
| Glycerin | 10.00% |
| Sorbitol | 17.00% |
| Sodium Laurel | 2.00% |
| Sulfate (SLS) | |
| Flavor | 1.00% |
| | 100.00% |

The hydrocolloids employed were the same as those employed in Example 2

Toothpaste compositions were prepared as follows:

### Process:

### Premix: Add Na MFP, Na Saccharin, Na Benzoate to DI Water

Add Hydrocolloid to glycerin in separate vessel, mix for 5 minutes using overhead mixer.
Add Sorbitol, continue to mix for 5 minutes.
Add water/salt premix, continue to mix for 25 minutes (at 65° C. for high viscosity carrageenan control; room temperature—about 25° C. for the other formulations)
Add this elixir phase mixture to Ross mixer, add Precipitated Calcium Carbonate, mix 20 minutes under vacuum.
Add SLS and flavor, mix 10 minutes under vacuum.

The compositions were stored at room temperature and at 50° C. for three months. The 0.1/sec shear rate was measured as described in Example 2. The results of such evaluation are presented in the Table below:

| | Room Temp. | | Flavor | 50° C. | | Flavor |
|---|---|---|---|---|---|---|
| Hydrocolloid | 3 Months | | Separation | 3 Months | | Separation |
| High Visc. | 402,000 | cps | none | 590,000 | cps | none |
| I-Carrageenan | | | | | | |
| 65° C. Processing | | | | | | |
| High Visc. | 252,000 | cps | none | 454,000 | cps | slight |
| I-Carrageenan | | | | | | |
| 25° C. Processing | | | | | | |
| 64:36 NaCa | 235,000 | cps | severe | 325,000 | cps | slight |
| Alginate | | | | | | |
| 85:15 NaCa | 258,000 | cps | severe | 296,000 | cps | slight |
| Alginate | | | | | | |
| High Visc. Na | 574,000 | cps | severe | 227,000 | cps | slight |
| Alginate | | | | | | |
| Ca Alginate | 248,000 | cps | severe | 450,000 | cps | slight |
| 33% 64:36 | 256,000 | cps | none | 387,000 | cps | none |
| NaCa Alginate | | | | | | |
| 33% Low Visc. | | | | | | |
| I-Carrageenan | | | | | | |
| 33% High Visc. | | | | | | |
| I-Carrageenan | | | | | | |

The above results demonstrate that the compositions of this invention provide desirable long term stability, even under elevated storage conditions. Further, the results indicate that desirable products can be obtained formulating the compositions of this invention at room temperatures, despite the presence of carrageenans which typically required the use of elevated temperatures.

What is claimed is:

1. A toothpaste composition comprising a binder composition wherein said binder composition comprises (a) alginate comprising a polyvalent cation alginate and (b) carrageenan comprising iota carrageenan.

2. The toothpaste composition of claim 1 wherein the polyvalent cation is calcium.

3. The toothpaste composition of claim 1 wherein the weight ratio of alginate to carrageenan in the binder composition is between 1:10 and 1:1.

4. The toothpaste composition of claim 3 wherein the weight ratio of alginate to carrageenan in the binder composition is between 1:3 and 1:1.

5. The toothpaste composition of claim 1 wherein the weight percent of polyvalent cation present in the alginate (a) is at least 10%, based upon the total weight of cations present in such alginate.

6. The toothpaste composition of claim 5 wherein the weight percent of polyvalent cation present in the alginate (a) is at least 20%, based upon the total weight of cations present in such alginate.

7. The toothpaste composition of claim 6 wherein the weight percent of polyvalent cation present in the alginate (a) is at least 30%, based upon the total weight of cations present in such alginate.

8. The toothpaste composition of claim 1 wherein the weight ratio of guluronic acid to mannuronic acid of alginate (a) is less than 1:1.

9. The toothpaste composition of claim 8 wherein the weight ratio of guluronic acid to mannuronic acid of alginate (a) is between 2:3 and 1:3.

10. The toothpaste composition of claim 1 wherein carrageenan (b) comprises kappa, kappa-2 or lambda carrageenan and at least 50% by weight of the total weight of carrageenan present is iota carrageenan.

11. The toothpaste composition of claim 1 wherein carrageenan (b) comprises 100% by weight iota carrageenan.

12. The toothpaste composition of claim 1 wherein such composition further comprises water and optionally further comprises one or more members of the group consisting of an abrasive, a humectant, a surfactant/foaming agent, a fluoride source, a sweetening agent, a flavor, a whitening agent, a preservative and a sensitivity agent.

13. The toothpaste composition of claim 12 wherein such composition comprises;
a) between 0.1% and 2% binder composition;
b) between 1% and 50% water;
c) between 0% and 50% abrasive;
d) between 0% and 40% humectant;
e) between 0% and 3% surfactant/foaming agent;
f) between 0% and 2% fluoride source;
g) between 0% and 1% sweetening agent;
h) between 0% and 2% flavor;
i) between 0% and 2% preservative; and
j) between 0% and 10% sensitivity agent;
wherein all such percentages are by weight based upon the total weight of the composition.
